(19) United States
(12) Patent Application Publication
(10) Pub. No.: US 2008/0014289 A1
(43) Pub. Date: Jan. 17, 2008

(54) Vehicle and method for treating and preventing acne vulgaris and exfoliating the skin hypohalous acids

(76) Inventor: Jianping Li, Bethesda, MD (US)

Correspondence Address: GERALD ANDERSON, 5615 MCLEAN DRIVE, BETHESDA, MD, 20814, US

(21) Appl. No.: 11/484,309

(22) Filed: Jul. 12, 2006

(51) Int. Cl. A61K 33/14; A61K 33/00

(52) U.S. Cl. 424/661; 424/723

(57) ABSTRACT

Vehicle and method is disclosed for treating and/or preventing skin disorders such as acne vulgaris and which exfoliates healthy skin by a topical application. The vehicle is a formulation incorporating hypohalous acid in a suitable pharmaceutical compound. The topical application comprises administering and scrubbing a therapeutically effective amount of hypohalous acid in the vehicle. The hypohalous acid may be hypochlorous acid, hypobromous acid, or hypoiodious acid. The suitable pharmaceutical vehicles include water, solutions, cleansers, lotion, cream, paper facial masks, and gels. The scrubbing action on the skin is exerted by a mechanic tool, such as hand, cloth towel, sponge, brush, and spraying device. Methods of preparing and compounding the vehicle for topical application and of its use are also set forth.

### FIELD OF THE INVENTION

Acne is a disease of the pilosebaceous unit of the skin appearing in males and females near puberty and usually becomes less active as adolescence ends. The intensity and duration of acne varies for each individual. Acne lesions are divided into noninflammatory and inflammatory lesions. Noninflammatory lesions consist of open and closed comedones. Inflammatory acne lesions are characterized by the presence of one or more of the following types: papules, pustules, nodules, and cysts.

The pathogenesis of acne is not completely understood. According to the most popular hypothesis, there are three pathogenic factors which cause acne:
1. The onset of puberty and hormonal imbalances cause increasing sebum production by the sebaceous gland (an oily secretion). Large amounts of sebum will build up on the surface of the skin and allow abnormal layers of cornified cell to adhere directly above the opening of the sebaceous gland duct (sometimes called retention keratosis). As a result, the secreted sebum plus excess cell debris form a plug (microcomedo) in the follicle canal.
2. The hair follicle canal is plugged by a protein known as keratin causing the formation of so-called whiteheads and blackheads while the sebaceous gland within the hair follicle continues to secret sebum. Sebum and dead cell debris in the sebaceous duct cannot exit to the surface of the skin thus accumulating in mass and causing the follicle to swell. If the swelling continues, the follicle may be ruptured, allowing the surface bacteria (normally found on the skin) to enter and cause local infections (e.g., zits or pimples). The zits in turn may enlarge and form cysts as bacteria multiply.
3. Proliferation of the bacteria Proprionibacterium acne in the sebaceous duct after the pilosebaceous follicle is plugged contributes to the process of acne formation. P. acnes is a normal skin resident and principal component of the microbic flora of the pilosebaceous follicle. Lipases, generated by P. acnes, hydrolyzes triglycerides of sebum to form free fatty acids which are primary irritants to the human immune system, and attract neutrophils to release hydrolases that weaken the wall of pilosebaceous follicle, the follicle wall thins, becomes inflamed (red papule), and ruptures. Comedones enter the dermis of the skin through the ruptures and produce an intense, foreign-body, inflammatory reaction that results in the formation of the acne pustule or cyst.

In response to these three most popular hypotheses, the current strategies of treating acne mainly target the aforementioned factors of the pathogenesis. Based on the mechanisms of their action, most anti-acne topical medicines can be divided into three groups:
1. Anti-inflammatory agents: the representative agents in the group are retinoids which are derivatives of vitamin A, such as Tretinoin, Adapalene and Tazarotene. These compounds can bind to nuclear retinoic acid receptors, affect expression of the genes involved in cell proliferation, cell differentiation and control local inflammatory reaction.
2. Keratolytic agents: this group of agents can promote desquamation of follicular epithelium and induce shedding of dry scale from the surface of the skin in an exfoliative-like fashion to inhibit comedonesis. Salicylic acid, α-Hydroxy acids (AHA) and azelaic acid are active ingredients used in these preparations;
3. Topical antibiotics: these antibiotics can decrease the population of P. acnes in the hair or pilosebaceous follicle and ultimately have an anti-inflammatory effect as well. Among the commonly used antibiotics are clindamycin and erythromycin.

### SUMMARY OF THE INVENTION

There are no medicines or preparations that treat or prevent acne by simultaneously acting upon all three of the pathogenic factors sited above. It is one object of the disclosed method to target on all these pathogenic factors simultaneously using a preparation of hypohalous acids.

The method being disclosed comprises a topical application of a preparation incorporating one of a group of hypohalous acids in a pharmaceutically acceptable vehicle. The group of hypohalous acids used in the treatment and/or prevention of acne vulgaris includes: hypochlorous acid, hypobromous acid and hypoiodious acid. The types of acne vulgaris that can be successfully treated and/or prevented with the disclosed method include noninflammatory lesions, including open and closed comedones and inflammatory lesions including papules, pustules, nodules and cysts. Examples of satisfactory pharmaceutical vehicles for the topical application of hypohalous acids include, but are not limited to, water, buffer solutions, cleansers, lotion, cream, facial masks and gels. The methods of preparing a preparation and its application are set forth.

The objectives of the present invention are to provide a topical application of an effective formulation in the treatment and/or prevention of acne vulgaris and related skin conditions, such as acne rosacea, and for the exfoliation of the skin for aesthetic effect. The formulation contains at least one active ingredient from a group of hypohalous acids including but not limited to hypochlorous acid, hypobromous acid and hypoiodious acid.

### DETAILED DESCRIPTION OF THE INVENTION

All oxyacids of halogen [fluorine (F), chlorine (Cl), bromine (Br), iodine (1), or astatine (At)] possess the general chemical formula HOX where X is the halogen atom. The group of hypohalous acids included hypochlorous acid (HOCl), hypobromous acid (HOBr) and hypoiodious acid (HOI) all exhibit similar chemical properties in that they are strong oxidants.

It is well known that Hypohalous acids can be manufactured from a variety of different materials, such as gas and inorganic substances. For example, HOCl and HOBr can be generated by the reaction between water and Cl₂ or Br₂ respectively:

Cl₂+H₂O⇄HOCl+HCl;

Br₂+H₂O⇄HOBr+HBr;

In addition, various compounds can be used to generate hypohalous acids, for instance, sodium hypochlorite (NaOCl), calcium hypochlorite [Ca(OCl)₂], lithium hypochlorite (LiOCl), sodium dichlor-s-triazinetrione (dichlor) and trichloro-s-triazinetrione (trichlor) etc. These compounds can be used to make hypochlorous acid when mixed with water. Traditionally, hypobromous acid and hypoiodious acid are produced from inorganic compounds such as sodium hypobromite (BrOCl) and potassium iodine (KI) respectively.

Chlorine is the most commonly used element for sanitation of drinking water. Bromine and Iodine can also be effectively used for the same purpose. All chlorine, regardless of whether it is introduced as a gas, as a liquid or as a dry compound, produces exactly the same biologically activity substance, hypochlorous acid (HOCl), when added to water. The balanced chemical equation between hypochlorous acid (HOCl) and hypochlorite ions (OCl⁻) in water is:

HOCl⇄H⁺+OCl⁻;

Hypochlorous acid (HOCl) is an active form of chlorine while the Hypochlorite ion (OCl⁻) is a relatively inactive form of chlorine in water. The sum of chlorine of Hypochlorous acid and hypochlorite ion in water is called Free Available Chlorine (FAC). The concentration of the active form (HOCl) is affected by the pH of the water to which it is added (see Table 1). For example, added to water having a pH of 8.0, it would take 2.5 ppm of FAC to provide about 0.5 ppm of HOCl; added to water having a pH of 7.5 about ½ (50%) of the FAC is in the active HOCl form and therefore it takes only 1.0 ppm of FAC to provide the same 0.5 ppm of HOCl.

TABLE 1
The relative percentage of HOCl and of OCl⁻ when
added to water having the specified initial pH levels.

| HOCl | Water | OCl⁻ |
|---|---|---|
| % of Chlorine as HOCl | pH | % chlorine as OCl⁻ |
| 90 | 6.5 | 10 |
| 73 | 7.0 | 27 |
| 66 | 7.2 | 34 |
| 45 | 7.6 | 55 |
| 21 | 8.0 | 79 |
| 10 | 8.5 | 90 |

The chemistry of bromine is similar in many respects to the chemistry of chlorine. When bromine is added to water, a chemical equilibrium between of hypobromous acid (HOBr) and hypobromite (OBr⁻) is formed. Like chlorine, the percentage of each form of Bromine is affected by pH of the solution. However, the effect is not as dramatic as it is with chlorine (see Table 2).

TABLE 2
The relative percentage of HOBr and of OBr⁻ when
added to water having the specified initial pH levels.

| HOBr | Water | OBr⁻ |
|---|---|---|
| % of bromine as HOBr | pH | % bromine as OBr⁻ |
| 100 | 6.0 | 0.0 |
| 99.4 | 6.5 | 0.6 |
| 98.0 | 7.0 | 2.0 |
| 94.0 | 7.5 | 6.0 |
| 83.0 | 8.0 | 17.0 |
| 57.0 | 8.5 | 43.0 |

Several chemical reactions involving hypohalous acids have been well documented by J. March^{[1]}. For example acyl hypohalites which are water-soluble can be formed in solutions of the hypohalous acids after adding carboxylic acids which are water-insoluble by halogenation reaction. They exist in equilibrium as following:

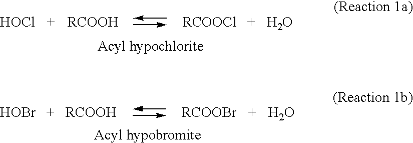

The addition reaction of hypohalous acids such as Hydroxy-chloro-addition to the double bonds of the following reaction equation (see Reaction 2) produces halohydrins. HOCl, HOBr, and HOI can be added to the double bonds in hydrophobic unsaturated fatty acids (water-insoluble) to produce hydrophilic halohydrins (water-soluble). Chlorohydrins are examples of the products of this reaction as shown by:

—CH═CH—+HOCl →—CHCl—CHOH— (Reaction 2)

### Hypohalous acids can exert its effect of treatment and/or prevention on acne vulgaris as well as aesthetic exfoliation depending on following aspects.

### Factor One: Dissolving Microcomedones:

According to J. J. White et al^{[2]}, the percent lipid composition of pooled human sebum is: ceramides (13%), fatty acid (47%), cholesterol (7%), cholesterol esters (2%), Squalene (11%), triglycerides (3%), and wax esters (17%). This lipid composition is the main component of microcomedones that cause acne to flare up. There is a large amount of fatty acid in the comedones which consists of saturated and unsaturated fatty acids. Among these fatty acids, palmitic acid is a saturated fatty acid and constitutes 25.3% of the total fatty acids and cis-Hexadec-6-enoic acid is an unsaturated fatty acid and constitutes 21.7% of the total fatty acids. The chemical structure of cis-Hexadec-6-enoic acid is shown below:

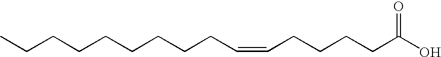

All fatty acids, both saturated and unsaturated, are hydrophobic and cannot be dissolved in water, but after being chemically modified by a halogenation reaction on the carboxyl group (—COOH) of the saturated fatty acids (See Reaction 1a and 1b), and by an addition reaction on the double bonds and a halogenation reaction on the carboxyl group of the unsaturated fatty acids (See Reaction 1a and 1b, Reaction 2), they are converted to hydrophilic compounds. For example, unsaturated fatty acid cis-Hexadec-6-enoic acid can conduct chlorination and an addition reaction by hypochlorous acid in water as following:

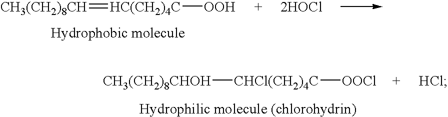

In a similar chemical reaction, hypochlorous acid can convert cholesterol into chlorohydrins, this results in hydrophobic cholesterol being converted to hydrophilic chlorohydrins. The chemical reaction is shown below:

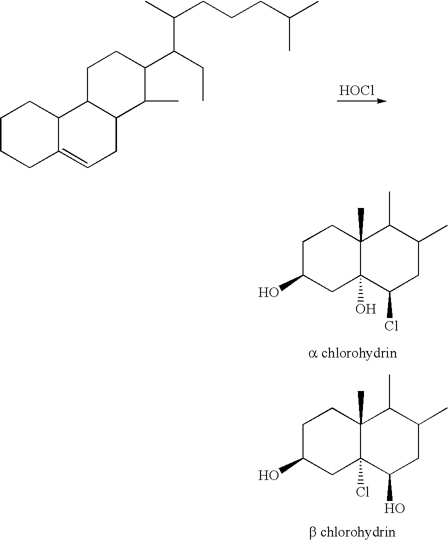

Thus we see that during the process of chemical halogenation and addition reaction in water, water-insoluble molecules, such as saturated fatty acid, unsaturated fatty acid and cholesterol, can be converted to water-soluble molecules, such as chlorohydrins. As the process proceeds using the disclosed method and preparation, the microcomedones which is blocking the follicular canal will be gradually dissolved and removed from the canal. Consequently one of the acne inducing factors of pathogenesis is resolved.

### Factor Two: Inducing Exfoliation of Scale from the Surface of the Skin:

The hypohalous acids are strong oxidants that react with many chemical function groups and biological molecules, such as amines, thiols, sulfides and disulfides, proteins, and unsaturated fatty acids. The reaction of HOCl with amino compounds leads to the generation of long-lived lipophilic derivatives of chloramines^{[3]} (see Reaction 3), such as monochloramine (NH₂Cl), mono-di-N-chloramines (RNHCl and RNCl₂), and other N—Cl derivatives.

HOCl+RNH₂→RNHCl+H₂O (Reaction 3)

Most of these derivatives appear to be reactive. The toxic effect of lipophilic derivatives such as NH₂Cl appears to be related to their ability to penetrate the hydrophobic barrier of cornified cells on the surface of skin and thus to oxidize and exfoliate the stratum corneum.

According to one research report^{[4]}, both HOCl and HOBr cause lysis of cells, and on an equimolar basis, HOBr is more efficient as a lytic agent by a factor of 10. When compared with the amount of oxidant required to cause lysis of unsaturated fatty acids, relatively high concentrations of HOCl are required compared to HOBr before a cholesterol halohydrin product was detectable.

### Factor Three: Inhibiting Proliferation of Bacteria:

Hypohalous acids are active, killing agents of bacteria and fungi. Hypochlorous acids easily enter micro organisms through their cell walls and destroy the sulfur groups on the cell's enzymes; this causes the cell's metabolism to stop, killing the organisms. They also break down cells through oxidizing fatty acids and protein in the cell membrane, resulting in the cell's death. Hypohalous acids can kill bacteria very quickly. The Association of Analytical Chemists confirms that 30 seconds is all that is needed to completely destroy a given concentration of bacteria in hot tubs, spas and swimming pools when disinfected by hypochlorous acid.

It is well known that the hypohalous acids can be produced in vivo by phagocytic cells^{[5]}. The cells, such as neutrophils, monocytes and eosinophils, contain peroxidases that catalyze the formation of hypohalous acids (HOX) from H₂O₂ and a halide (X⁻). Myeloperoxidase, the enzyme present in neutrophils and monocytes will utilize Cl⁻, Br⁻, although Cl⁻ is the preferred substrate. Under physiological conditions, utilization of Cl⁻ results in the production of hypochlorous acid. The enzyme Eosinophil peroxidase on the other hand does not react readily with Cl⁻. Under physiological conditions, this enzyme uses Br⁻ to form hypobromous acid. For example, neutrophils respond to phagocytic and inflammatory stimuli by producing superoxide (O₂⁻) and H₂O₂, and by releasing granule proteins either into the phagosome or extracellularly, myeloperoxidase (MP³⁺), the most abundant granule enzyme in phagocytic cells, catalyses the conversion of H₂O₂ and Cl⁻ to HOCl.

MP³⁺+H₂O₂→MP³⁺H₂O₂

MP³⁺H₂O₂+Cl⁻→MP³⁺+HOCl+OH⁻

### HOCl destroys many harmful organisms, including bacteria, fungi etc., efficiently in vivo by this mechanism.

Therefore, hypohalous acids can cure acne efficiently by combining these three functions simultaneously: dissolving microcomedones to clear any plugging of the follicle canal, inducing exfoliation of scale from the surface of the skin by removing the cornified cells which adhere to the surface of the skin, and inhibiting proliferation of P. acnes to avoid inflammation. At low concentration and in pharmaceutical acceptable vehicles, a hypohalous acid exerts an exfoliation style action on the outer most layers of the skin. As reaction 3 shows, hypohalous acid can be reacted with the amino group of biological molecules leading to the generation of chemical reactive lipophilic derivatives, i.e. chloramines, these lipophilic derivatives penetrate the hydrophobic barrier of cornified cells on the surface of skin and thus to oxidize stratum corneum resulting in exfoliation.

In the preferred embodiments the formulations of the present method may be in the form of water, surfactants, emulsifiers, thickeners, emollients, and humectants. Hypohalous acid can be added into personal-care products such as facial washing solution, cleanser, paper facial mask, lotion, cream and gel as an active ingredient along with a pharmaceutically acceptable carrier or a vehicle. The following specific examples are intended to be illustrative of the invention and should not be construed as limiting the scope of the invention as defined by appended claims.

No matter what kind of halogen compounds for preparing the formulations, the active ingredient is hypohalous acids (HOX) in the final preparations, it may be either hypochlorous acid (HOCl) if the compounds being used are sodium hypochlorite, calcium hypochlorite, lithium hypochlorite, sodium dichloro-s-triazinetrione dihydrate (dichlor) or trichloro-s-triazinetrione; hypobromous acid (HOBr) if the compounds being used are sodium bromide or magnesium bromide; mixture of hypochlorous acid and hypobromous acid if the compounds being used are bromine-chlorine-dimethylhydantoine (BCDMH); or hypoiodious acid (HOI) if the compound being used is potassium iodine (KOI).

### 1. Washing Solution:

Water is used as a pharmaceutical vehicle of hypohalous acid in this solution for treating and/or preventing acne vulgaris, and exfoliation. In order to be stable for long shelf life, the washing solution is prepared from two stock solutions, stock solution A and stock solution B, the solution is prepared freshly by mixing specific amount of each stock solution into defined amount of water before the treatment being exercised. Stock solution A contains an amount of active ingredient-hypohalous acid; stock solution B is pH modifier, it is either acid such as HCl or base such as NaOH depending on what the compound is used for the preparation of stock solution A. In some cases, stock solution B even can be omitted from the washing solution preparation if pH value of stock solution A is in the range of 6.5˜7.5. It is also possible to mix stock solution B into stock solution A as one stock solution.

Accordingly, the anti-acne activity of halogen compounds depend on the concentration of hypohalous acid present in washing solution after mixing stock solution A and stock solution B. Stock solution B adjusts pH value of the washing solution to optimal range, pH6.5˜7.5, for maximizing the concentration of the hypohalous acid and the effects on anti-acne.

When using chlorine compounds to prepare the washing solution, it can be seen from the Table 1, that when the pH value of the solution is above 7.5, very little (<50%) FAC exists as active HOCl while most becomes inactive OCl⁻. If pH value of the solution is below 6.0, noxious chlorine gas (Cl₂) is formed and the preparation does not serve as an effective anti-acne agent. Thus, for preparation of a washing solution, it is important to maximize HOCl concentration and minimize all other forms of chlorine by adjusting pH value. For effective treatment, it is highly desirable to keep the pH of the washing solution between 6.5 and 7.5 to ensure adequate HOCl (45˜90% of FAC) activity without forming chlorine gas. Although the concentration of HOCl in a washing solution with a pH the range 6.5 to 7.5 can gradually decrease as the FAC falling due to chlorine gas evaporation during the treatment, the change shouldn't be significant in the short time required for treatment (10 to 30 minutes). If the pH range is 6.0 to 8.5 washing solution still exerts the desired effect but treating period has to be extended.

A preclinical trial showed that the optimal range for the concentration of HOCl in the washing solution at the beginning of treatment is between 0.95 and 28 ppm with a pH value of about 7.0 and a temperature range of 25° C.˜60° C. The concentration of HOCl in the washing solution may be higher than this value depending on where the washing solution is to be applied, e.g. when treating a patient's back for trunk acne, the concentration of HOCl in the washing solution may be as high as 60 ppm for maximum effect in the short term because the thickness of skin on this section of a body is around 3 mm (as compared to a skin thickness of 0.1 mm at the chin section of the face).

To prepare stock solution A in which hypochlorous acid is active ingredient, different amount of inorganic or organic compounds may be used. Common compounds for preparing hypochlorous acid solution are sodium hypochlorite (NaOCl), calcium hypochlorite [Ca(OCl)₂], lithium hypochlorite (LiOCl), sodium dichloro-s-triazinetrione dihydrate (dichlor), and trichloro-s-triazinetrione (trichlor).

### EXAMPLE 1A

### Stock solution 1Aa: sodium hypochlorite solution, FAC 12% (Sigma-Aldrich, MO, USA)

### Stock solution 1 Ab: 1.2 N Hydrochloric Acid.

Preparation: Add 8.24 mL of 37% hydrochloric acid (Mallinckrodt, MO. USA) to 100 mL D. I. H₂O.

### Preparation of Washing Solution

Add 2.5 mL of stock solution 1Aa and 5 mL of stock solution 1 Ab into 2 gallons (7.57 L) warm tap water (30° C. to 50° C.). In this pH 7 washing solution, the concentration of NaOCl is 29 ppm, and the concentration of HOCl is 13.7 ppm right after preparation. As chlorine gas evaporated during the treatment, the FAC and concentration of HOCl in the washing solution decreases. The pH value may vary from 6.5 to 7.5 depending on the pH value of tap water used for the preparation.

### EXAMPLE 1B

### Stock solution 1 Ba: sodium hypochlorite solution, FAC 6.15% (Clorox, CA. USA)

### Stock solution 1 Bb: 1.2 N Hydrochloric Acid.

Preparation: Add 8.24 mL of 37% hydrochloric acid (Mallinckrodt, MO. USA) to 100 mL D. I. H₂O.

### Preparation of Washing Solution

Add 5 mL of stock solution 1 Ba and 5 mL of stock solution 1 Bb into 2 gallons (7.57 L) warm tap water (30° C. to 50° C.). In this pH 7 treating solution, the concentration of NaOCl in the washing solution is 29 ppm, and the concentration of HOCl is 13.7 ppm right after the preparation.

### EXAMPLE 1C

### Stock solution 1 Ca: calcium hypochlorous acid, FAC 12%.

Preparation: add 18 g of calcium hypochlorite, granular (FAC 65%) to 100 mL D. I. H₂O, mix until dissolved completely.

### Stock solution 1 Cb: 1.2 N Hydrochloric Acid.

Preparation: Add 8.24 mL of 37% hydrochloric acid (Mallinckrodt, MO. USA) to 100 mL D. I. H₂O.

The method of preparing washing solution is similar to example 1A.

### EXAMPLE 1D

### Stock solution 1 Da: lithium hypochlorous acid, FAC 12%.

Preparation: add 34 g of lithium hypochlorite (powder, FAC 35%) to 100 mL D. I. H₂O, mix until dissolved completely.

### Stock solution 1 Db: 1.2 N hydrochloric acid.

Preparation: Add 8.24 mL of 37% hydrochloric acid (Mallinckrodt, MO. USA) to 100 mL D. I. H₂O.

### The method of preparing washing solution is similar to example 1A.

### EXAMPLE 1E

### Stock solution 1 Ea: sodium dichloro-s-triazinetrione, FAC 12%.

Preparation: add 21.3 g of sodium dichlor (sodium dichloro-s-triazinetrione, chemical formula NaCl₂C₃N₃O₃, FAC 56%) to 100 mL D. I. H₂O.

### Preparation of Washing Solution

Since Stock solution is neutral (pH 6.9), there is no requirement of secondary stock solution to adjust pH. Only add 2.5 mL of stock solution 1 Ea to 2 gallons (7.57 L) warm tap water (30° C. to 50° C.).

### EXAMPLE 1F

### Stock solution 1 Fa: trichloro-s-triazinetrione, FAC 12%.

Preparation: add 13 g of Trichlor (Trichloro-s-triazinetrione, chemical formula Cl₃C₃N₃O₃, FAC 90%) to 100 mL D. I. H₂O, mix until dissolved completely.

### Stock solution 1 Fb: 0.25 N Sodium hydroxide.

Preparation: add 1 g of NaOH to 100 mL D. I. H₂O.

### Preparation of Washing Solution

The method of preparing this washing solution is similar to example 1A. Using bromine compounds to prepare the washing solution, in which hypobromous acid is an active ingredient, the most common bromide compounds used are sodium bromide and bromine-chlorine-dimethylhydantoine (BCDMH). The formal chemical name of BCDMH is 1-Bromo-3-Chloro-5,5-Dimethyl Hydantoin (Formula: C₅H₆BrClN₂O₂). The halogen content is 63%. BCDMH is a powder granular organic substance. After it is dissolved in water hypobromous acid and hypochlorous acid are released.

Like chlorine, the chemical form of bromine in the solution is determined by pH value of the solution (see Table 2). When the pH value is below 8.7, more hypobromous acid (HOBr) is formed, when pH value is above 8.7, hypobromite ions will be more abundant. Interestingly, comparing the concentrations of hypochlorous acid with hypobromous acid at pH 7.0 in the solution, 73% of FAC exists as HOCl, and 27% of FAC is OCl⁻; but 98% of bromine existing as HOBr, only 2% of bromine is OBr⁻. Accordingly, less amount of bromine compound is required for preparing the washing solution even hypobromous acid is slightly less effective than hypochlorous acid in anti-acne activity. The optimal concentration range of bromine for anti-acne is 0.5 ppm to 5 ppm in pH 7.0 solution.

### EXAMPLE 1G

### Stock solution 1 Ga: 25 g/L sodium bromide;

### Stock solution 1 Gb: 0.1 N sodium hydroxide (NaOH);

### Preparation of Washing Solution

Add 2 mL of stock solution 1 Ga and 2 mL of stock solution 1 Gb into 2 gallon (7.57 L) warm tap water (30° C. to 50° C.). In this formulation, concentration of bromine is 4 ppm, and the concentration of hypobromous acid is around 3.94 ppm (pH around 7.0) right after preparation.

### EXAMPLE 1H

### Stock solution 1 Ha: Bromine-chlorine-dimethylhydantoine (BCDMH).

Preparation: 3g of BCDMH dissolve into 100 mL of D. I. H₂O.

### Stock solution 1 Hb: 0.1 N NaOH.

The method of preparing washing solution is similar to example 1G.

The chemical properties of Iodine are similar to chlorine and bromine, but its reaction is usually less violent. To prepare the washing solution in which hypoiodious acid (HOI) is active ingredient, the most common iodine compound used in the present invention is potassium iodine (KI). The optimal concentration range of iodine for present invention is 0.1 ppm to 0.5 ppm in pH 7.0 solution.

### EXAMPLE 1I

### Stock solution 1Ia: 5 g/L Potassium Iodine.

### Preparation of Washing Solution

Add 1 mL of stock solution 1Ia into 2 gallons (7.57 L) warm tap water (30° C. to 50° C.). The concentration of iodine ion is 0.5 ppm. Since hypohalous acid is not stable in the solution, the washing solution has to be prepared freshly before the treatment. The method of using washing solutions which are prepared in example 1A-1I as followed.

### Treatment Using a Washing Solution

Treatment is divided into two stages, treatment period and maintenance period. User should conduct the treatment daily during treatment period until no acne break out; then can switch to maintenance period.

### Primary Treatment Period (daily treatment):

1. Wash the face regularly with soap completely removing oil and grime from the surface of the skin. Concentrate on areas of the skin prone to suffering from acne, such as forehead, chin, jawline, around the lips and nose.
2. Prepare the washing solution as indicated in Examples 1A-1I.
3. Submerge your face fully into the washing solution, in order to extend time for skin contacting with the solution, moving the head as much as practical while submerged. You can extend the time of submersion by:
   —exhaling slowly while submerged;
   —lift the head out of water and breathe in and then submerge again.
Repeated submersions improve the effectiveness of the treatment and may be repeated as many as 30 times.
(Note: There is mild exfoliation effect of the solution, in order to avoid over exfoliating thin sections of the skin after repeated treatment, wearing goggles and applying Vaseline is recommended. The thickness of the eyelid is only 0.01 mm compared to 0.1 mm at forehead, and wearing goggles will protect this part of the face. Rubbing some Vaseline on the ridge of nose, nostrils and lips is recommended for similar reasons).
4. Scrub the skin affected by acne with a towel, preferable a cloth towel soaked in the washing solution. Mainly focus on the areas of the skin most affected by acne but scrubbing the whole face is recommended since it is not easy to predict where acne will flare up. For optimal results this stage should take around 5 minutes.
(Note: step 4 is a key to the treatment of skin actively affected by acne with the washing solution, it is necessary to pay a great attention on the scrubbing movement).
Repeat step 3 and step 4 once or twice depending on the skin condition.
5. Drain the solution, wash whole face with clean water and apply cosmetic products as necessary, such as a moisturizer.

### Secondary Treatment Period

Periodic treatment for maintenance and prevention begins if there is no new acne out breaks for one week during the Primary Treatment Period. Daily treatments are not usually necessary during this period. The interval between treatments is dependent on an individual user's skin condition. Preclinical experience suggests that if acne recurres within three days after ending treatment, then treatment should begin again and be repeated every two days; if acne recurres within four days after stopping treatment then the treatment should begin again and be repeated every three days. Similar periodic intervals can be estimated based on the response of the individuals skin to the treatment. All steps in the Primary Treatment Period should be repeated during the Secondary Treatment Period.

### Exfoliation Using a Washing Solution

A washing solution can be used as an aesthetic exfoliating solution. The procedure of usage is similar with Primary Treatment Period of treating acne, and it can be practiced once or twice per week depending on individual user's skin condition.

### 2. Cleanser

Hypohalous acid may be included as an active ingredient in a cleanser. All hypohalous acids are inorganic compounds and can be mixed with hydrophilic substances if there is no reaction between them. The anti-acne and exfoliating function of a selected hypohalous acid works efficiently and conveniently in a cleanser formulation including any pharmaceutically acceptable surfactants, emulsifiers, humectants, thickeners, as inactive ingredients as long as there is no reaction between the inactive ingredients and the selected hypohalous acid. The compositions for a cleanser in present method may also contain other inactive ingredients that are commonly included in topical pharmaceutical compositions such as preservatives, binders, fragrance and opacifiers.

Surfactants are compounds which are both hydrophilic and lipophilic. The hydrophilic portion in the surfactant molecule may be either a salt group which ionizes in solution to yield a charged, anionic or cationic group, or a polar moiety such as a hydroxyl group (OH), which can easily form a hydrogen-bond to enhance water solubility of entire molecule. The oil-soluble portion of surfactant molecule is general a hydrocarbon, usually a relatively long, branched or straight carbon chain. Surfactants can bring water-soluble hypohalous acid molecule to the water-insoluble microcomedones on the surface of the skin where the unsaturated fatty acids and cholesterol which are the main components in comedones can be oxidized and dissolved. Such surfactant include polyethylene sorbitan esters, polyoxyethylene & polyoxypropylene esters, polyethylene glycol esters, polyoxyethylene caster oil derivatives, alkyl phenol ethoxylates, fatty esters and glyceryl esters, lanolin and lanolin products, fatty alcohols, which are stable in the presence of strong oxidative agents such as hypohalous acids, e.g., polysorbates derivatives (Tween −20, −40, −80); ethoxylated derivatives of hydrogenated sterols, such as lanolin; taurates, such as sodium methyl cocoyl taurate. The preferred surfactant is a polyethoxylated sterol derivative, e.g. lanolin and taurates such as sodium methyl cocoyl taurate.

The amount of surfactant present in the cleanser composition may vary from 1% to 95% by weight of the composition. Preferably, the surfactant is present in an amount from 5% to about 20% by weight.

Thickeners are used in topical cleanser compositions and in the present invention any pharmaceutical acceptable thickener which is stable in oxidizing solution can be used in the cleanser composition. Examples of acceptable thickeners are clay, polyethylene glycols (PEGs) and their derivatives, cetostearyl alcohol, microcrystalline cellulose, powdered cellulose and cellulose derivatives, starch and starch derivatives. The thickeners may be present in the compositions of the invention in an amount from 0.5% to 10% by weight of compositions. Preferably, the thickener is a mixture of cellulose derivatives and clay and present in total amount from about 1% to about 10% by weight.

### EXAMPLE 2A

A washing solution is prepared as in examples 1A to 1I but using cold D. I. H₂O in stead of warm tap water to which 5% of sodium methyl cocoyl taurate 1% of clay and 1% of cellulose derivative are added, adjust pH of the solution to between 6.5 to 7.5 by 1.2 N HCl. These ingredients are mixing intensively, packaged in a non-transparent air-tight container and stored at room temperature.

### EXAMPLE 2B

A washing solution is prepared as in example 1A to 11 but using cold D. I. H₂O in stead of warm tap water, adding 5% of sodium methyl cocoyl taurate, 3% of clay, 2% cellulose derivative, 2% Aloe vera gel, and 1% Tween 20, adjust pH to 6.5 to 7.5 by 1.2 N HCl. The ingredients are mixing gently, packaged in a non-transparent air-tight container and stored at room temperature.

Anti-acne activity of hypohalous acid in washing solution may be more efficient than in cleanser when the concentration of active ingredient is similar. It may therefore be necessary to increasing the concentration of the hypohalous acid in the cleanser to produce similar results.

### EXAMPLE 2C

### Stock solution 2 Ca: Sodium hypochlorite solution, FAC 12% (Sigma-Aldrich, MO, USA)

### Stock solution 2Cb, 2.4 N Hydrochloric Acid.

### Preparation: Add 19.8 mL of 37% hydrochloric acid (Mallinckrodt, MO. USA) to 100 mL D. I. H₂O.

### Preparation of Washing Solution

Add 5 mL of stock solution 2 Ca and 5 mL of stock solution 2 Cb into 2 gallons D. I. water (7.57 L). This is a pH 7 washing solution and the concentration of NaOCl is 58 ppm, and the concentration of HOCl is 27 ppm. This washing solution is then intensively mixed with 5% of Sodium methyl cocoyl taurate, 25% of cetostearyl alcohol intensively, packaged in a non-transparent air-tight container, and stored at room temperature.

The treatment using the cleanser is as follows:

Approximately 5 mL to 10 mL of the cleaner prepared in Example 2A-2C are applied on a wet cloth towel, sponge or brush, then scrubbed with moderate strength, for 2 minutes. This step is followed by a rinse with clean water. Then apply fresh cleanser on the cloth towel and scrub. The process is repeated two or three times. This treatment is to be preformed once daily during the primary treatment period as in the washing solution treatment. After acne is under control, the treatment might be adjusted to twice per week depending on the users acne condition.

The exfoliation using the cleanser is as follows:

Cleanser can be used as aesthetic exfoliating preparation, the procedure of usage is similar to the treatment using the cleanser, it can be practice once per week.

### 3. Paper Facial Mask

Paper may be used as a convenient vehicle for the active ingredient-hypohalous acid solution. Any thick papers which can absorb large volume of water are suitable. The paper is cut to a size and shape suitable for use as a facial mask. Preparation of washing solution is similar to example 1A to 11, soak paper facial mask into the solution and apply it to the face.

### 4. Gel

Gel can be also used as a convenient vehicle for the active ingredient-hypohalous acids. The substance used for the gel should be inert to hypohalous acid. For example cellulose derivatives which are stable in oxidative solution. Preparation of a washing solution is similar to example 1A, 3% VEEGUM HS 12.5% cetostearyl alcohol are mixed gently, packaged in a non-transparent air-tight container, and stored at room temperature.

It will be apparent to those skilled in the art that the foregoing describes the preferred embodiments of the invention, both as to their methods and formulations and use, and particularly for providing a simple method of formulating, including at least a hypohalous acid, and applying a topical application to the skin for the treatment/prevention of acne and for the exfoliation of the skin. It will be understood that the present invention is intended to embrace not only the methods, structures, materials, procedures and formulations aforesaid, but also all reasonable equivalents thereof as understood by those skilled on the art and limited only by the appended claims.

What is claimed:

1. A method of treating and/or preventing human acne vulgaris and of skin exfoliation comprises: preparing a vehicle including a therapeutically effective amount of hypohalous acid suitable for topical application, and pH value of the vehicle is between 6.0 to 8.5, and applying the vehicle to the skin of a patient with acne vulgaris.

2. A method as defined in claim 1, wherein the said hypohalous acid includes at least one of the group hypochlorous acid, hypobromous acid or hypoiodious acid.

3. A method as defined in claim 2 wherein the said hypohalous acid is hypochlorous acid present in an amount ranging from 0.5 ppm to 100 ppm by free available chlorine (FAC).

4. A method as defined in claim 2 wherein the said hypohalous acid is hypobromous acid present in an amount ranging from 0.5 ppm to 50 ppm by bromine.

5. A method as defined in claim 2 wherein the said hypohalous acid is hypoiodious acid present in an amount ranging from about 0.1 ppm to 20 ppm by iodine.

6. A method as defined in claim 2 wherein the said hypohalous acid is the mixture of hypochlorous acid and hypobromous acid.

7. A method as defined in claim 1 wherein said vehicle is selected from the group consisting of water, solutions, suspensions, cleansers, lotion, cream, paper facial mask, and gels.

8. A method as defined in claim 1 wherein the application of the vehicle to the skin includes a scrubbing action wherein the patient scrubs the section of skin suffering acne vulgaris.

9. A method as defined in claim 8 wherein the patient scrubs the section of skin suffering acne vulgaris a mechanic tools drawn from a group of tools including a cloth towel, a sponge or a brush.

10. A method of exfoliating human skin comprising the steps of administering a aesthetically effective amount of hypohalous acid in a vehicle suitable for topical application to the skin and scrubbing the areas of the skin most needing exfoliation.

11. A method as defined in claim 10, wherein the said hypohalous acid includes at least one of the group hypochlorous acid, hypobromous acid or hypoiodious acid.

12. A method as defined in claim 10, wherein the said vehicle is selected from the group consisting of water, solutions, suspensions, cleansers, lotion, cream, paper facial mask, and gels.

13. A method as defined in claim 10 wherein the said scrubbing includes the step of scrubbing the skin with a tool, such as a cloth towel, a sponge, a brush or a spraying device.

14. A vehicle for treating and/or preventing acne vulgaris and for exfoliating skin said vehicle used as a topical application and including a therapeutically or aesthetically effective amount of a hypohalous acid.

15. The vehicle as in claim 14 wherein said hypohalous acid is selected from a group including hypochlorous acid, hypobromous acid or hypoiodious acid.

16. The vehicle as in claim 14 wherein said vehicle is provided in a form suitable for topical application selected from the group consisting of water, solutions, suspensions, cleansers, lotion, cream, paper facial mask, and gels.

17. The vehicle as in claim 16 wherein said form of said vehicle is a cleanser and further includes hypohalous acid present in an amount ranging from 0.1 ppm to 100 ppm of the halogen component of the acid.

18. The vehicle as in claim 16 wherein said cleanser further includes a surfactant present in an amount ranging from 1% to 95% by weight of the composition and a thickener present in an amount ranging from 0.5% to 20% by weight of compositions.
